(19) United States
(12) Patent Application Publication
(10) Pub. No.: US 2016/0113501 A1
(43) Pub. Date: Apr. 28, 2016

(54) Functional Magnetic Resonance Imaging (fMRI) Methodology Using Transverse Relaxation Preparation and Non-Echo-Planar Imaging (EPI) Pulse Sequences

(71) Applicants: THE JOHNS HOPKINS UNIVERSITY, Baltimore, MD (US); KENNEDY KRIEGER INSTITUTE, INC., Baltimore, MD (US)

(72) Inventors: Jun Hua, Woodstock, MD (US); Craig Kenneth Jones, Owings Mills, MD (US); Qin Qin, Ellicott City, MD (US); Peter VanZijl, Ellicott City, MD (US)

(21) Appl. No.: 14/895,609

(22) PCT Filed: Jun. 3, 2014
(86) PCT No.: PCT/US2014/040603

§ 371 Date: Dec. 3, 2015

Related U.S. Application Data
(60) Provisional application No. 61/830,360, filed on Jun. 3, 2013.

(51) Int. Cl. A61B 5/00

(52) U.S. Cl. CPC A61B 5/0042; A61B 2576/026

(57) ABSTRACT

An embodiment in accordance with the present invention provides a new acquisition scheme for T2-weighted BOLD fMRI. It employs a T2 preparation module to induce the BOLD contrast, followed by a single-shot 3D fast gradient echo (GRE) readout with short echo time (TE<2 ms). The separation of BOLD contrast generation from the readout substantially reduces the “dead time” due to long TE required in spin echo (SE) BOLD sequences. This approach termed “3D T2prep-GRE,” can be implemented with any magnetic resonance imaging machine, known to or conceivable by one of skill in the art. This approach is expected to be useful for ultra-high field fMRI studies that require whole brain coverage, or focus on regions near air cavities. The concept of using T2 preparation to generate BOLD contrast can be combined with many other fast imaging sequences at any field strength.

### CROSS REFERENCE TO RELATED APPLICATIONS

This application claims the benefit of U.S. Provisional Patent Application No. 61/830,360 filed on Jun. 3, 2013, which is incorporated by reference, herein, in its entirety.

### GOVERNMENT SUPPORT

This invention was made with government support under NIH RO15P41-RR015241 awarded by the National Institutes of Health. The government has certain rights in the invention.

### FIELD OF THE INVENTION

The present invention relates generally to imaging. More particularly the present invention relates to a system and method for magnetic resonance imaging.

### BACKGROUND OF THE INVENTION

High field [7 Tesla (T)] human MRI scanners have become available in recent years with the promise of an approximately linear increase in signal-to-noise ratio (SNR) with field strength. In addition, high field is particularly attractive to blood-oxygenation-level-dependent (BOLD) functional MRI (fMRI) as the BOLD contrast shows a supra-linear increase with field strength. To date, the majority of BOLD fMRI experiments are performed using gradient echo (GRE) echo-planar-imaging (EPI) sequences. While they provide excellent sensitivity to signal changes during functional stimulation with high acquisition efficiency, they often suffer from geometric distortions and signal dropouts in regions near air cavities such as the orbitofrontal cortex and temporal lobes, which are exacerbated at high field. Spin echo (SE) based sequences are useful alternative approaches to alleviate these problems. More importantly, the T2-weighted contrast in SE BOLD is more specific to the site of neuronal activity at high field than the T2*-weighted contrast in GRE BOLD, making it an appealing option for brain mapping at high field. SE BOLD fMRI can be performed using approaches such as fast spin echo (FSE), gradient spin echo (GRASE), stimulated echoes, balanced and non-balanced steady state free precession (SSFP), RASER, and most commonly, SE EPI. One of the main constraints for SE sequences, however, is the high power deposition imposed mainly by the large number of refocusing radiofrequency (RF) pulses, which unfortunately scales with the square of the field strength.

More importantly, the T2 or T2* contrast in most BOLD fMRI methods is generated during the imaging sequence, which may impose some intrinsic constraints. For instance, a long echo time (TE) is required for SE BOLD, which produces some “dead time” that limits the acquisition efficiency and temporal resolution for fMRI. Alternatively, T2 contrast can be induced with driven equilibrium (DE, also known as driven equilibrium Fourier transform or DEFT). In MRI, driven equilibrium was originally used to enhance SNR for SE sequences with short repetition time (TR). It was also applied in GRE sequences as a preparation module immediately before the readout train, referred to as T2 preparation or T2-prep. Early examples of applying this concept include methods that combine T2 preparation with a segmented 3D fast GRE readout for T2-weighted anatomical imaging in the brain and liver. Such T2-prepared segmented 3D fast GRE sequences have also been used to improve the contrast between blood and tissue in cardiac imaging and peripheral angiography, to detect myocardial perfusion changes, in dynamic susceptibility contrast (DSC) cardiac MRI, and for myelin water quantification. T2 preparation can also be combined with other imaging sequences. For fMRI in the brain, a 3D T2prep-EPI sequence was proposed to combine T2 preparation with a 3D EPI readout for mixed T2- and T2*-weighted BOLD fMRI.

It would therefore be advantageous to provide a new method for acquiring whole brain fMRI images with minimal distortion and dropouts.

### SUMMARY OF THE INVENTION

The foregoing needs are met, to a great extent, by the present invention, wherein in one aspect a method for magnetic resonance imaging of a subject includes employing a T2-weighted preparation module to induce blood-oxygenation-level-dependent (BOLD) contrast. The method includes providing a single-shot, fast-gradient echo (GRE) readout. The method also includes acquiring an image of the subject.

In accordance with an aspect of the present invention, the method includes providing the single-shot fast-GRE readout having a short echo time and using the short echo time of approximately <2 ms. The method includes using the single shot fast-GRE readout taking the form of at least one of turbo field echo, TFE, or turbo flash. The method includes acquiring the image in the form of a whole brain fMRI image with minimal distortion and dropouts and acquiring the image with a spatial resolution of approximately mm isotropic. Additionally, the method includes acquiring the image having a temporal resolution of 2.3 s at 7 T. The BOLD contrast is generated before providing the single-shot, fast-gradient echo (GRE) readout. Two 180° pulses in the T2-weighted preparation module can be used to compensate for phase variations and to suppress inflow effects. A spoiler gradient can be played at an end of the T2-weighted preparation module on a first phase encoding axis that has a lowest gradient duty cycle to dephase any residual transverse magnetization. A SINC RF pulse can be used for refocusing, and the single-shot fast-gradient echo readout can have low-high (centric) phase encoding.

In accordance with another aspect of the present invention, a system for magnetic resonance imaging includes a magnetic resonance imaging scanner. The system includes a non-transitory computer readable medium programmed to execute steps. The steps include employing a T2-weighted preparation module to induce blood-oxygenation-level-dependent (BOLD) contrast. The steps also include providing a single-shot, fast-gradient echo (GRE) readout and acquiring an image of the subject.

In accordance with another aspect of the present invention, the non-transitory computer readable medium is integrated into the magnetic resonance imaging scanner. Alternately, the non-transitory computer readable medium resides on a computing device networked with the magnetic resonance imaging scanner.

In accordance with yet another aspect of the present invention, the steps include providing the single-shot fast-GRE readout having a short echo time and using the short echo time of approximately <2 ms. The steps include using the single shot fast-GRE readout taking the form of at least one of turbo field echo, TFE, or turbo flash. The steps include acquiring the image in the form of a whole brain fMRI image with minimal distortion and dropouts and acquiring the image with a spatial resolution of approximately 2.5 mm isotropic. Additionally, the steps include acquiring the image having a temporal resolution of 2.3 s at 7 T. The BOLD contrast is generated before providing the single-shot, fast-gradient echo (GRE) readout. Two 180° pulses in the T2-weighted preparation module can be used to compensate for phase variations and to suppress inflow effects. A spoiler gradient can be played at an end of the T2-weighted preparation module on a first phase encoding axis that has a lowest gradient duty cycle to dephase any residual transverse magnetization. A SNC RF pulse can be used for refocusing, and the single-shot fast-gradient echo readout can have low-high (centric) phase encoding.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a pulse sequence of 3D T2prep-GRE, according to an embodiment of the present invention. A T2 preparation module (90° x-180° y-180° y-90° -x, spatially nonselective; hyperbolic secant adiabatic pulses were used for 180° pulses) was applied immediately before the readout. Two 180° pulses were used in T2 preparation to compensate phase variations and to suppress inflow effect. A spoiler gradient was played at the end of T2 preparation on the first phase encoding axis that has the lowest gradient duty cycle to dephase any residual transverse magnetization. A single-shot 3D fast GRE readout with low-high (centric) phase encoding was used. TR_{GRE}: time period between two consecutive echoes during the fast GRE readout; TE_{GRE}: echo time for one echo in 3D fast GRE; TR: time period between two consecutive 3D fast GRE readout; TE: duration of T2 preparation excluding the spoiler at the end.

FIG. 2 illustrates a comparison of image quality for MPRAGE (anatomical, voxel=1×1×2.5 mm³, 55 slices, reconstructed from the original 1 mm isotropic scan), 3D T2prep-GRE fMRI scan (TR=2.3 s, 2.5 mm isotropic voxel, 55 slices) and 2D multi-slice SE EPI (TR=9 s, 2.5 mm isotropic voxel, 55 slices, no functional stimulation). Due to SAR limits, 2D SE EPI has to use a TR 4 times longer than 3D T2prep-GRE to acquire the same number of slices covering the whole brain. Sagittal, coronal and 3 axial slices at different locations (slice number 12, 26 and 47) are shown. Geometric distortion and signal dropouts are visible in SE EPI images, especially in the frontal and temporal lobes (red arrows), but are minimal in 3D T2prep-GRE images. S: superior; P: posterior; L: left.

FIGS. 3A-3C illustrate representative fMRI results from one subject. More particularly, FIG. 3A illustrates an fMRI activation map with 3D T2prep-GRE (TR=2.3 s, 2.5 mm isotropic voxel, 55 slices) and FIG. 3B illustrates a fMRI activation map with 2D multi-slice SE EPI (TR=2.3 s, 2.5 mm isotropic voxel, 17 slices). In both FIGS. 3A and 3B, voxels meeting activation criteria are highlighted with their t-scores (scale indicated on the right). No spatial smoothing was performed in the analysis. FIG. 3C illustrates a graphical representation of average time courses from voxels meeting activation criteria in visual (red, x-mark) and motor (green, open circle) cortex with 3D T2prep-GRE, and in visual cortex with 2D SE EPI (blue, diamond). In visual cortex, only common voxels activated in both scans were included. A separate fMRI scan was performed using the same 3D fast GRE readout without T2 preparation, and the average time course from this scan (black, square) was calculated over voxels activated in the previous 3D T2prep-GRE scan (both visual and motor cortex). Four blocks were averaged to one block. The two vertical dashed lines indicate the start and cessation of stimulus. The error bars represent inter-voxel standard deviations within subject, which are much larger than the inter-subject standard deviations reported in Table 2.

FIGS. 4A and 4B illustrates images of representative temporal SNR (tSNR) efficiency maps from one subject. FIG. 4A illustrates a 3D T2prep-GRE (TR=2.3 s, mm isotropic voxel, 55 slices). FIG. 4B illustrates a 2D SE EPI (TR=2.3 s, mm isotropic voxel, 17 slices, angled to cover more cortex and to avoid orbitofrontal cortex.

FIG. 5 illustrates images of 3D T2prep-GRE fMRI images and activation maps with TR=1860 ms, voxel=1.5×1.5×1.6 mm³ and 84 slices from one subject. Voxels meeting activation criteria are highlighted with their t-score (scale indicated on the right, threshold=2.3).

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a new acquisition scheme for T2-weighted BOLD fMRI. It employs a T2 preparation module to induce the BOLD contrast, followed by a single-shot 3D fast gradient echo (GRE) readout with short echo time (TE<2 ms). The separation of BOLD contrast generation from the readout substantially reduces the “dead time” due to long TE required in spin echo (SE) BOLD sequences. This approach termed “3D T2prep-GRE,” can be implemented with any magnetic resonance imaging machine, known to or conceivable by one of skill in the art. This approach is expected to be useful for ultra-high field fMRI studies that require whole brain coverage, or focus on regions near air cavities. The concept of using T2 preparation to generate BOLD contrast can be combined with many other fast imaging sequences at any field strength.

According to a method of the present invention, the T2 contrast is created using a T2 preparation module, followed immediately by a single-shot 3D fast GRE, which is known as turbo field echo, TFE, or TurboFLASH, readout sequence with short TE (<2 ms). Such a readout has much less geometric distortion and fewer signal dropouts than EPI as well as low power deposition, and is commonly used in high-resolution anatomical scans such as the Magnetization Prepared RApid Gradient Echo (MPRAGE) sequence. Using this 3D T2prep-GRE approach, whole brain fMRI images with minimal distortion and dropouts can be acquired with a spatial resolution of 2.5 mm isotropic (55 slices) at a temporal resolution of 2.3 s at 7 T. Human fMRI experiments with simultaneous flashing checkerboard and bilateral finger tapping were performed to evaluate the 3D T2prep-GRE approach and compare it with the conventional 2D multi-slice SE EPI sequence.

A new T2-weighted BOLD fMRI pulse sequence, 3D T2prep-GRE, which consists of a T2 preparation module to create SE BOLD contrast followed by a single-shot 3D fast GRE readout with short TEGRE, is introduced. The “decoupling” of BOLD contrast generation from the readout sequence substantially reduces the “dead time” due to long TE required in SE BOLD and gives more freedom to choose various readout sequences. Compared with the widely used 2D multi-slice SE EPI sequence, the main advantages of the 3D T2prep-GRE approach include: minimal geometric distortion across the whole brain as well as lower SAR, allowing greater spatial coverage and tSNR and CNR efficiency. 3D fast GRE readout with short TEGRE is less sensitive to magnetic susceptibility variations than EPI, and is commonly used in high-resolution anatomical imaging sequences such as MPRAGE. The readout in 3D T2prep-GRE is similar to that in MPRAGE, resulting in fMRI images that resemble anatomical images, which makes spatial alignment easier than for EPI images with nonlinear distortion. One of the major factors that limit spatial coverage in SE EPI is power deposition. This is less of a concern for 3D T2prep-GRE, mainly because only two refocusing 180° pulses are deployed in each TR and small flip angle (4°) RF pulses are used in the readout train. The 3D readout also permits parallel imaging in two phase encoding directions, rather than one in the case of 2D SE EPI, which can be used to further improve acquisition efficiency. As demonstrated here, 55 slices could be acquired with 3D T2prep-GRE while 2D SE EPI could cover merely 17 slices with the same TR, spatial resolution and SAR level (Table 1). When averaged over commonly activated voxels in the visual cortex, tSNR was 11% lower in 3D T2prep-GRE, mainly due to the small flip angle used in the readout and the high SENSE factor in two directions. However, its tSNR efficiency was 60% higher than 2D SE EPI (Table 2). In ROI based analysis, the tSNR difference between the two methods was minimal, while tSNR efficiency in 3D T2prep-GRE was 92% greater.

TABLE 1
3D T2prep-GRE and 2D multi-slice SE EPI pulse sequences.

| | | | slice | | fat |
|---|---|---|---|---|---|
| | TR(s) | voxel size | number | SAR¹ | suppression |
| 3D | 2.3 | 2.5 mm | 55 | 74% | Not needed² |
| T2prep- | | isotropic | | | |
| GRE | | | | | |
| (fMRI) | | | | | |
| 2D SE | 2.3 | 2.5 mm | 17 | 75% | No³ |
| EPI | | isotropic | | | |
| (fMRI) | | | | | |
| 2D SE | 9 | 2.5 mm | 55 | 75% | Yes |
| EPI | | isotropic | | | |
| (at | | | | | |
| rest) | | | | | |

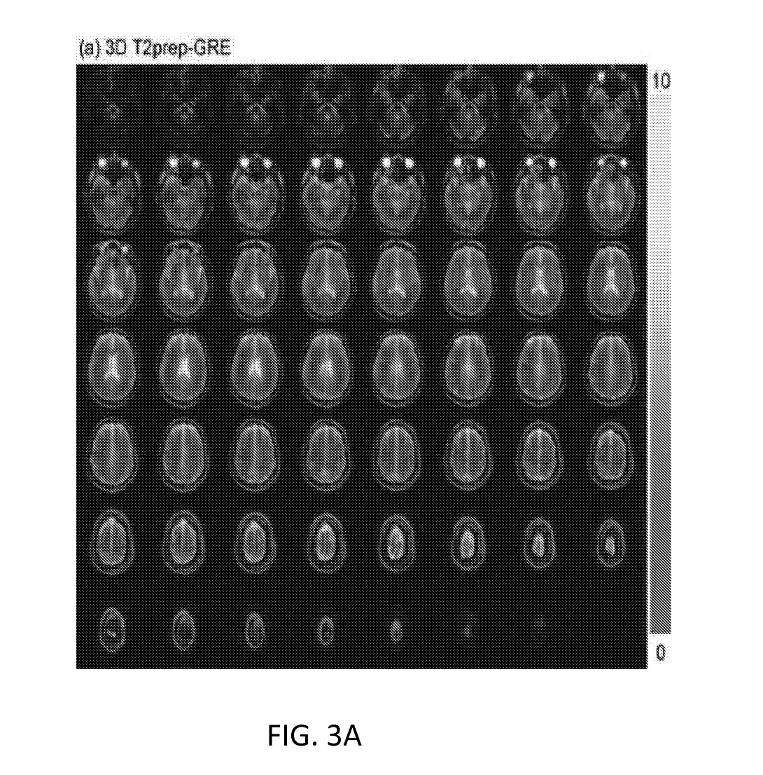

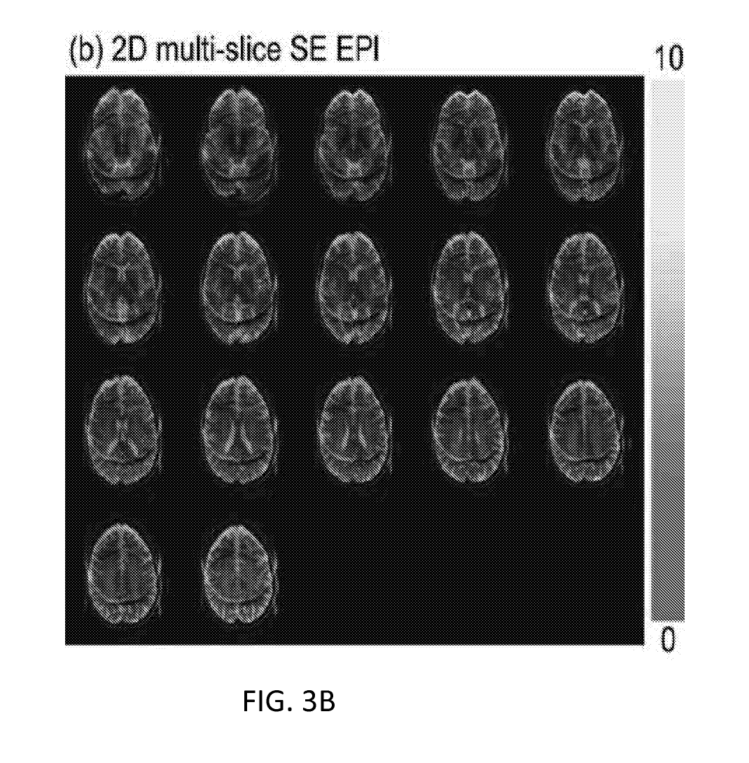

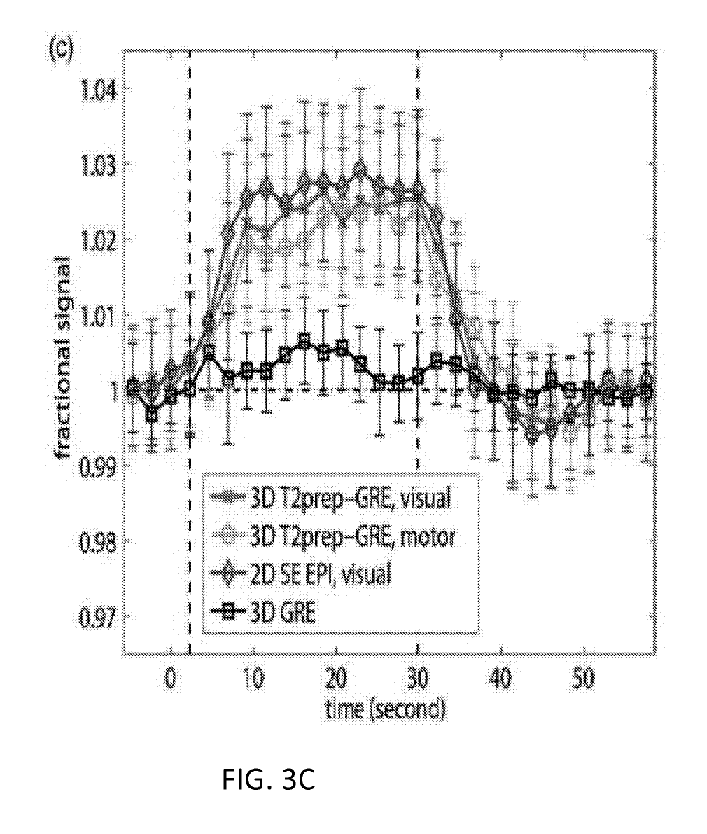

¹Specific absorption rate (SAR) shown on the scanner, approximately 2.4 W/kg, 74-75% of the maximum SAR approved by FDA.
²3D fast GRE images have few fat shift artifacts in general (FIG. 3A).
³Fat suppression will significantly increase SAR. With the same TR, spatial resolution and SAR level, if fat suppression would be applied, the number of slices allowed would decrease to 14.

TABLE 2
Summary of fMRI results from all subjects.

| | | | | | | | | |
|---|---|---|---|---|---|---|---|---|
| | | Voxel | ΔS/S | | tSNR | | CNR | |
| Shim | Cortex | Numbers | (%) | tSNR | efficiency | CNR | efficiency | t-score |

Averaged Over Activated Voxels (in visual cortex, voxels activated in all scans)-without spatial
smoothing

| | | | | | | | | | |
|---|---|---|---|---|---|---|---|---|---|
| 3D | Volume^{d} | Visual | 340 ± 124 | 2.5 ± 0.3 | 58 ± 10 | 284 ± 30 | 1.4 ± 0.2 | 6.5 ± 0.8 | 3.3 ± 0.1 |
| T2prep- | Volume | Motor | 304 ± 134 | 2.3 ± 0.7 | 56 ± 13 | 271 ± 43 | 1.1 ± 0.1 | 5.2 ± 0.5 | 3.1 ± 0.1 |
| GRE^{b} | | | | | | | | | |
| 2D SE | Volume | Visual | 422 ± 181 | 2.9 ± 0.7 | 65 ± 16 | 177 ± 41 | 1.7 ± 0.2 | 4.8 ± 0.5 | 3.6 ± 0.2 |
| EPI^{c} | High | Visual | 424 ± 170 | 2.9 ± 0.6 | 65 ± 15 | 177 ± 42 | 1.7 ± 0.2 | 4.8 ± 0.4 | 3.6 ± 0.2 |
| | Order^{c} | | | | | | | | |

Averaged Over Activated Voxels (in visual cortex, voxels activated in all scans-with spatial
smoothing (5 mm FWHM Gaussian kernel)

| | | | | | | | | | |
|---|---|---|---|---|---|---|---|---|---|
| 3D | Volume | Visual | 397 ± 152 | 2.4 ± 0.3 | 60 ± 11 | 292 ± 33 | 1.4 ± 0.2 | 6.5 ± 0.8 | 3.4 ± 0.2 |
| T2prep- | Volume | Motor | 341 ± 163 | 2.3 ± 0.6 | 57 ± 13 | 275 ± 42 | 1.1 ± 0.2 | 5.2 ± 0.6 | 3.1 ± 0.2 |
| GRE | | | | | | | | | |
| 2D SE | Volume | Visual | 498 ± 226 | 2.7 ± 0.7 | 64 ± 18 | 175 ± 47 | 1.6 ± 0.2 | 4.7 ± 0.6 | 3.6 ± 0.2 |
| EPI | High | Visual | 485 ± 222 | 2.7 ± 0.7 | 66 ± 19 | 178 ± 52 | 1.6 ± 0.3 | 4.7 ± 0.7 | 3.6 ± 0.2 |
| | Order | | | | | | | | |

Averaged over ROIs (GM voxels in primary visual and motor cortex, respectively)

| | | | | | | | | | |
|---|---|---|---|---|---|---|---|---|---|
| 3D | Volume | Visual | 1485 ± 267 | 0.7 ± 0.1 | 51 ± 6 | 247 ± 14 | 0.4 ± 0.1 | 1.7 ± 0.4 | 0.8 ± 0.3 |
| T2prep- | Volume | Motor | 1046 ± 295 | 0.6 ± 0.2 | 48 ± 7 | 236 ± 20 | 0.3 ± 0.1 | 1.3 ± 0.6 | 0. ± 0.3 |
| GRE | Volume | Visual | 1458 ± 267 | 0.8 ± 0.5 | 47 ± 11 | 128 ± 31 | 0.4 ± 0.2 | 0.9 ± 0.4 | 0.8 ± 0.3 |
| 2D SE | High | Visual | 1458 ± 267 | 0.8 ± 0.6 | 45 ± 10 | 123 ± 30 | 0.4 ± 0.3 | 0.9 ± 0.5 | 0.8 ± 0.3 |
| EPI | Order | | | | | | | | |

1. ^{a}Mean values ± standard deviations over all subjects (n = 5). Definitions of ΔS/S, tSNR, tSNR efficiency, CNR, and CNR efficiency are described in the Methods section.
2. ^{b}fMRI scan (a).
3. ^{c}fMRI scan (b).
4. ^{d}Volume shim over a 120 × 120 × 50 mm3 (AP × RL × FH) volume centered on the brain was applied in these scans to achieve a reasonably homogeneous field (B0) across the entire brain. A water line width of <60 Hz was achieved.
5. ^{e}Optimal high order shim in the visual cortex (AP × RL × FH = 40 × 120 × 50 mm3) using a localized shimming tool. A water line width of <60 Hz was achieved. Similar results were also obtained with optimal high order shim in the entire brain (data not shown).

2D multi-slice SE EPI is by far the most commonly used sequence for T2-weighted SE BOLD fMRI. A long echo train is often needed, in which only the echo at TE is perfectly refocused. This introduces some additional T2* weighting in the MR signals, which causes geometric distortion and results in a larger relative signal change (ΔS/S) during functional activation than expected with a pure T2-weighted SE BOLD. 3D fast GRE with long TEGRE has been used for T2*-weighted GRE BOLD especially in many early fMRI studies. Here, in 3D T2prep-GRE, the shortest possible TEGRE (usually <2 ms) was used in the GRE readout allowing us to minimize T2* effects. This was demonstrated with fMRI experiments using 3D fast GRE without T2 preparation, which had few activated voxels in the brain and relative signal changes (ΔS/S) that were not significantly different from baseline when averaged over activated voxels in fMRI scans with T2 preparation (FIG. 3C). The time course and ΔS/S for 3D fast GRE without T2 preparation in FIG. 3C did show a slight positive trend (albeit not statistically significant) during activation, which suggests that there may be still some residual T2* effects induced by the 3D fast GRE readout even with very short TEGRE. Note that this may be an overestimate of T2* effects in the actual T2prep-GRE sequence, as the T2 preparation module will eliminate most intravascular (due to short blood T2 at 7 T) and extravascular BOLD effects around veins, leaving only the extravascular BOLD effects around capillaries due to dynamic averaging to be detected by the following GRE readout. Besides, the underlying mechanisms of the T2* effects in these two sequences are different. In SE EPI, it stems from the echoes acquired at times other than TE that are not perfectly refocused, leading to different T2* effect for each echo, thus varying T2* contamination for different spatial frequency. On the other hand, the T2* effect in 3D fast GRE is the result of free induction decay, which is the same for each echo and independent of spatial frequency, and can be minimized by using shortest TEGRE. Further investigation is warranted to discern these details as how they affect the BOLD contrast in these methods.

The number of activated voxels and ΔS/S (thus CNR and t-score) were all slightly lower in 3D T2prep-GRE than 2D SE EPI, while the CNR efficiency was 35% higher in 3D T2prep-GRE when averaged over commonly activated voxels (Table 2). In ROI based analysis, the differences in ΔS/S, CNR and t-score between the two methods were minimal, while the CNR efficiency was 88% higher in 3D T2prep-GRE (Table 2). The smaller ΔS/S in 3D T2prep-GRE may be attributed to two main factors. First, it may be partially the result of smaller T2* contamination and purer T2-weighted BOLD signals as discussed above. Second, as two refocusing pulses were used in the T2 preparation module (double echo CPMG), the effective T2, thus optimal TE for BOLD contrast, is expected to be longer than a conventional SE EPI sequence with one refocusing pulse. At 7 T, the intravascular BOLD effects are negligible due to very short blood T2 values. The extravascular BOLD effects around veins should be largely refocused in SE sequences. Therefore, the dominant contribution to SE BOLD contrast at 7 T comes from the extravascular BOLD component around capillaries (dynamic averaging). It is estimated that the equivalent TE to induce the same ΔS/S in a double echo CPMG sequence is approximately 80 ms, as compared to 50 ms in a single SE sequence. This means that the same TE of 50 ms used for both sequences here may lead to a smaller ΔS/S in 3D T2prep-GRE. Thus, using an optimal TE may increase ΔS/S in 3D T2prep-GRE. Note that this potential requirement for longer TE (not TEGRE) in 3D T2prep-GRE will only increase its total TR by 30 ms or so. Meanwhile, as physiological noise is dominant in fMRI, the MR signal loss due to a longer TE might only lead to a slight decrease in tSNR. Further investigation is required to compare single and double SE BOLD contrasts, and to determine the optimal TEs experimentally.

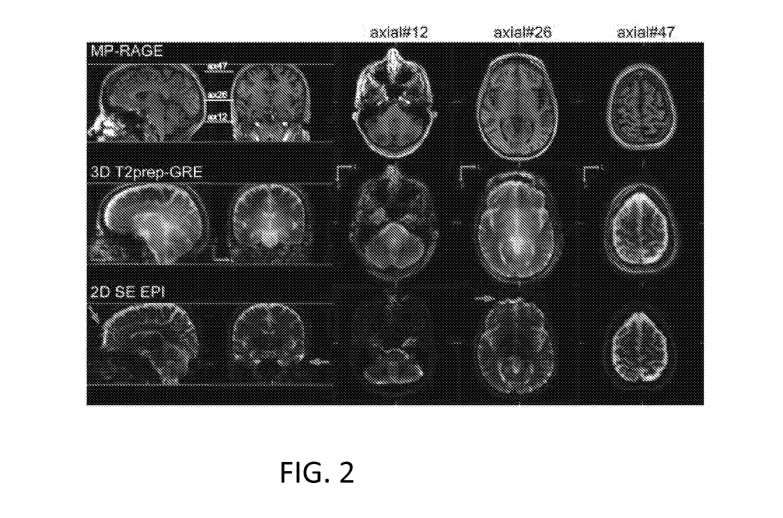

Crusher gradients surrounding the refocusing pulses can be applied in T2 preparation to alleviate problems arising from RF pulse imperfections in T2 preparation caused mainly by B1 field inhomogeneity. On the other hand, it was also suggested that the key to eliminate this problem is to design more robust RF pulses, as crusher gradients can only prevent interference between the residual transverse magnetization and subsequent pulse sequence, but cannot restore the signal loss from inaccurate RF pulse flip angles. In this study, dielectric bags were inserted between subjects' head and coil to improve B1 homogeneity, and optimized adiabatic 180° pulses that can tolerate a large variation (>50%) in B1 were used in T2 preparation. However, there still appeared to be some B1 inhomogeneity (hyper-intensity in the middle of the brain in FIG. 2). This could be caused mainly by the 90° pulses in T2 preparation and the readout RF pulses. While these artifacts should not undermine the main conclusions in this study, it is important to apply crusher gradients, design RF pulses with enhanced B1 tolerance and use advanced B1 shimming techniques to improve the accuracy of T2 preparation in future studies. When adding crusher gradients in T2 preparation, it is also important to consider gradient moment nulling (first order) or velocity compensation to suppress motion related artifacts. Here, as the duration of T2 preparation (50 ms) is short compared with typical echo train length in sequences such as FSE and GRASE, artifacts due to subject motion are perhaps negligible. Also, considering the very short blood T2 at 7 T and long CSF T2, flow related artifacts stemming from T2 preparation are probably minor. In addition, if needed, for instance, when imaging brain regions close to large blood vessels or ventricles, the motion-sensitized driven equilibrium (MSDE) approach (different type of crusher gradients applied in T2 preparation) can be used to minimize confounding signals from fast flowing spins. The MSDE approach can also suppress the inflow effect when very short TRs are used.

Volume shim, which is now widely available on MRI scanners, was used in all scans to compare images under the same B0 shim condition. Nevertheless, it should be noted that while 3D T2prep-GRE images are less sensitive to field inhomogeneity, the geometrical distortion in EPI images can be substantially reduced with more advanced B0 shim techniques. The SE EPI fMRI scans are repeated with optimal high order shim in the visual cortex using a localized shimming tool. The tSNR/CNR results in the visual cortex were similar to those obtained with volume shim (Table 2). This can perhaps be explained by the fact that the occipital lobe was sufficiently well shimmed in both methods with volume shim already, as shown by the image quality in the visual regions in FIGS. 2, 3A-3C, and 5.

The bulk of the power deposition (SAR) in SE sequences comes from the refocusing 180° RF pulses. Therefore, the main reason that 3D T2prep-GRE has lower SAR provided the same 180° pulses are applied is that it only needs two 180° pulses in each volume TR, while the number of 180° pulses in 2D SE EPI is determined by the number of slices and usually far exceeds two. Here, a SNC 180° pulse was used in SE EPI, while a hyperbolic secant adiabatic 180° pulse, which has a higher SAR but a better B1 inhomogeniety tolerance, was used in T2 preparation (details in Methods). As demonstrated above, the SAR level was still much lower in T2prep-GRE. Besides, the flip angles of the excitation RF pulses in T2prep-GRE are also much smaller than those in SE EPI (4° and 80° here, respectively), which further lowers the SAR. It should be noted that SAR can be reduced by applying RF pulses with longer duration and lower peak B1, and/or variable rate (VR) pulses (also known as variable rate gradient (VRG), or variable rate selective excitation (VERSE) pulses), thus improving spatial coverage for SE sequences.

Geometic distortion is a well-known problem for EPI that has been studied extensively. Advanced B0 shim techniques can improve global B0 field homogeneity. Several approaches have been proposed for local distortion correction in EPI, such as methods based on anatomical images, B0 field maps, point spread function maps, and others. While these approaches can significantly reduce geometric distortion in EPI, most of them require extra scan time for reference images and sometimes prolonged computational time. Moreover, head motion during fMRI scans may cause nonlinear dynamic changes of field susceptibility thus distortion during a fMRI run. Parallel imaging and multiband techniques can subtantially shorten the echo train in EPI readout, which also mitigates geometric distortion at some expense of SNR. Therefore, a fMRI scan with less intrisic distortion, such as the 3D T2prep-GRE method, may be useful in certain applications.

One confounding factor of the 3D T2prep-GRE sequence is that its signal intensity varies during k-space acquisition mainly due to T1 relaxation. This is inherent to all magnetization prepared 3D fast GRE sequences such as MPRAGE, which will lead to spatial blurring/smoothing that deteriorates the spatial resolution and artificially enhances the SNR. Furthermore, for 3D T2prep-GRE, T1 relaxation during the readout echo train will also lower the T2 contrast between baseline and activation for fMRI. As a centric phase encoding profile was used here, the T2-weighted BOLD contrast for higher spatial frequencies may be diminished. The T1 relaxation during readout will also introduce some T1-weighting in T2prep-GRE images, but as T1 change is relatively small during functional activation and T1 values become longer and converge (smaller relative difference) at higher fields, this effect should have small influence on the BOLD contrast. This confounding issue can be alleviated by using k-space filtering or variable flip angle in the readout echo train. Further investigation is needed to improve this aspect of the T2prep-GRE sequence.

It should be noted that the 3D GRASE sequence is another promising approach and has been gaining popularity for SE BOLD fMRI. 3D GRASE was also implemented on the 7 T scanner. With the same TR=2.3 s, SAR level <77% (2.5 W/kg), slightly shorter TE=40 ms due to additional signals from stimulated echoes and other parameters identical, 3D GRASE could accommodate 44 slices with the same spatial resolution, slightly less than 3D T2prep-GRE (55 slices). Further investigation is merited for a detailed comparison between these two sequences to characterize their sensitivity (tSNR and CNR), specificity and contrast mechanisms, and to find suitable applications for fMRI.

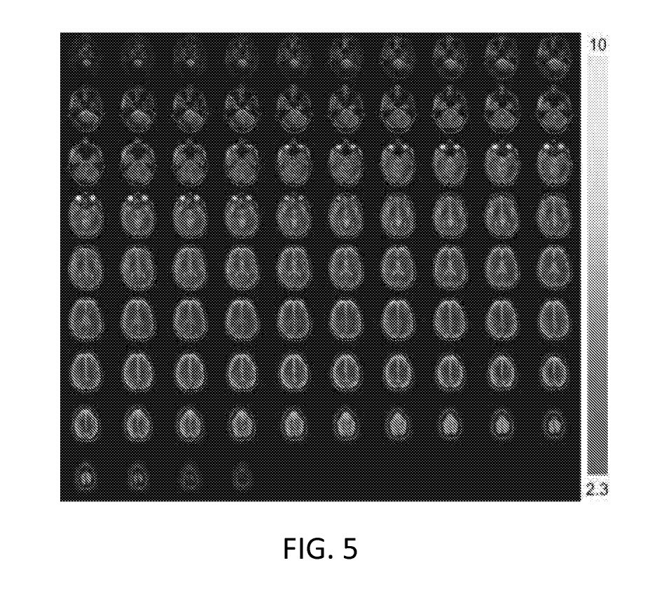

A voxel size of mm isotropic was used in this proof-of-concept study to demonstrate the principle of the 3D T2prep-GRE method for whole-brain coverage, and to compare it with 2D SE EPI with the same spatial and temporal resolution. The 3D T2prep-GRE method can also be used in fMRI studies focusing on certain regions of the brain, in which case much finer spatial resolution can be obtained with localized coverage. In additional, the 3D T2prep-GRE method can be further expedited using techniques such as partial Fourier sampling and multiband. The multiband technique can substantially speed up many MRI sequences. Using power independent of number of slices (PINS) multiplexing 2D SE EPI at 7 T, whole-brain coverage can be achieved with 84 slices of 1.6 mm thickness, 1.5 mm in-plane resolution and a TR of 1860 ms using a four-fold multiband acceleration. With the proposed 3D T2prep-GRE sequence, identical temporal (TR) and spatial (voxel) resolution and coverage using a partial Fourier fraction of ⅝ (typical value for fMRI, other parameters same, centric encoding) were achieved without multiplexing. Robust activation was detected in the brain with this sequence for a single subject (FIG. 5). The main difference here is that in most SE BOLD approaches such as SE EPI and GRASE, the extent to which their acquisition efficiency can be improved by partial Fourier methods and parallel imaging is limited by the long TE required for T2 contrast, whereas it is no longer a constraint in the readout of 3D T2prep-GRE. One drawback, though, for partial Fourier methods and parallel imaging is that they may have higher SNR penalties than the multiband technique. Nevertheless, the 3D T2prep-GRE sequence may also be further accelerated using the multiband technique in a way similar to 3D multi-slab GRASE. Further development is needed to investigate and compare SNR penalties and other characteristics of these sequences.

An important goal for SE BOLD fMRI at ultra-high field is to simultaneously achieve sub-mm spatial resolution, whole-brain coverage, and TRs of 1-2 s or less. This is still not possible with current SE BOLD methods, including the proposed approach. One way to obtain higher spatial resolution is to reduce the field of view. T2prep-GRE can achieve sub-mm resolution with partial brain coverage (less slices, and/or smaller field of view). Several SE EPI BOLD studies in human brain have demonstrated sufficient sensitivity to detect neuronal activity with sub-mm resolution in a single slice (e.g., 0.5×0.5×3 mm3 voxel, TR=6 s; 0.5×0.5×1 mm3 voxel, TR=2 s). As T2prep-GRE is shown to have comparable tSNR/CNR and greater tSNR/CNR efficiency than SE EPI at mm isotropic voxel size, it is reasonable to expect that T2prep-GRE would also have sufficient sensitivity to detect typical SE BOLD signal changes at sub-mm resolution. Many exciting new technologies are being developed to further improve MRI acquisition efficiency, such as the improvement of multi-channel receiving coils to accelerate parallel imaging as well as the multiband technique. These methods would also greatly benefit T2prep-GRE, and can potentially be combined to further improve its efficiency and sensitivity.

### EXAMPLE

An exemplary implementation of the present invention is described herein, in order to further illustrate the present invention. The exemplary implementation is included merely as an example and is not meant to be considered limiting. Any implementation of the present invention on any suitable subject known to or conceivable by one of skill in the art could also be used, and is considered within the scope of this application.

Five healthy human subjects, who gave written informed consent before participating in this Johns Hopkins Institutional Review Board (IRB) approved, Health Insurance Portability and Accountability Act (HIPAA)-compliant study, were scanned on a 7 T Philips MRI scanner (Philips Healthcare, Best, The Netherlands). A 32-channel phased-array head coil (Nova Medical, Wilmington, Mass.) was used for RF reception and a head-only quadrature coil for transmit. Two rectangular pads (23×10×2 mm) filled with high dielectric constant materials were placed between the lateral sides of the subjects' head and the coil to improve field homogeneity. fMRI sessions were performed using visual stimulation with blue/yellow flashing checkerboard (36.8 s off/27.6 s on, 4 repetitions, 1 extra off period in the end) delivered using a projector from the back of magnet. The subjects were instructed to perform bilateral finger tapping during the flashing periods. Each fMRI run took 4 min and 54.4 s during which 128 image volumes (TR=2.3 s) were acquired.

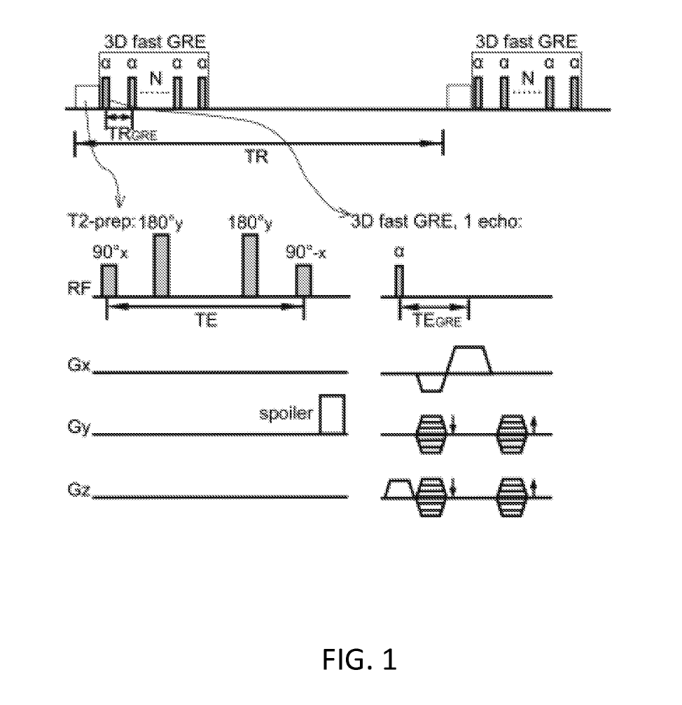

Three pseudo-randomized fMRI scans were performed on each subject: (a) 3D T2prep-GRE (illustrated in FIG. 1): 55 slices, single shot 3D fast GRE readout, TR_{GRE} (this is the TR between two echoes during the fast GRE readout)/TE_{GRE}=3.6/1.6 ms, flip angle (FA)=4°, readout duration=1916 ms, turbo direction=radial (k-space traversed in radial scheme), parallel imaging acceleration (SENSE factor)=3×3(APxFH), partial Fourier fraction =1×1(APxFH, i.e., no partial Fourier here), low-high (centric) phase encoding. A T2 preparation module (90° x-180° y-180° y-90° -x, duration or effective TE=50 ms, spatially nonselective; hyperbolic secant adiabatic pulses were used for 180° pulses, duration=15 ms, bandwidth=1050 Hz, peak B1=15 μT, and >95% inversion at 50% B1) was applied immediately before the readout. The same 90° and 180° RF pulses optimized for 7 T were used in T2 preparation. Two 180° pulses were used in T2 preparation to compensate phase variations and to suppress inflow effects. A spoiler gradient was played at the end of T2 preparation on the first phase encoding axis that has the lowest gradient duty cycle to dephase any residual transverse magnetization. (b) 2D multi-slice SE EPI: 17 slices with interleaved order, no gap between slices, TE=50 ms, single-shot multi-slice SE EPI, FA=70° (smaller than the Ernst angle (approximately 110°) to reduce power), SENSE factor=3, partial Fourier fraction=⅝(AP), fat suppression. A SNC RF pulse (duration=5.38 ms, bandwidth=816 Hz, peak B1=15 μT) was used for refocusing. (c) Same as (a) but without the T2 preparation to test whether there are residual BOLD effects induced by the readout. Common parameters in (a-c): field of view (FOV)=210×210 mm², voxel size=mm isotropic, TR (TR between two consecutive scans)=2.3 s. Note that due to the specific absorption rate (SAR) limit, 2D SE EPI can only accommodate fewer than ⅓ of the slices allowed in 3D T2prep-GRE. To compare image quality in the whole brain, another 2D SE EPI scan (d) was performed without functional stimulation: 55 slices, a long TR of 9 s, with fat suppression, and other parameters identical to fMRI scan (b). The parameters of the 3D T2prep-GRE and 2D SE EPI sequences are compared in Tablet To demonstrate the potential to be further accelerated, another 3D T2prep-GRE scan (e) was performed on one subject with the same functional paradigm: voxel=1.5×1.5×1 6 mm³, 84 slices, TR_{GRE}/TE_{GRE}=3.1/1.4 ms, readout duration=1674 ms, TR=1860 ms, partial Fourier fraction=(⅝)×(⅝)(APxFH), other parameters same as scan (a). High-resolution anatomical images were acquired using MPRAGE (voxel=1 mm isotropic, TR/TE/inversion time (TI)=4.0/1.9/563 ms, SENSE factor=2×2). Volume shim over a 120×120×50 mm³ (APxRLxFH) volume centered on the brain was applied in all scans to achieve a reasonably homogeneous field (B0) across the entire brain. As EPI is much more sensitive to susceptibility-induced B0 field inhomogeneity, the SE EPI fMRI scan (b) was also repeated with optimal high order shim in the whole brain (over the same volume as the volume shim), and in the visual cortex only (AP×RL×FH=40×120×50 mm³), using the localized shimming tool developed by Schar et al. In both cases, a water line width of <60 Hz was achieved.

Data analysis was carried out using the Statistical Parametric Mapping (SPM8, University College London, UK) software package and several in-house Matlab R2009b (Mathworks, Natick, Mass.) routines. Preprocessing steps for fMRI images include realignment to correct for subject motion during the scans, detrending, slice timing correction for 2D multi-slice SE EPI (not needed for 3D scans), co-registration between fMRI and anatomical images, and segmentation to get grey matter (GM) masks. No spatial smoothing was applied in the fMRI analysis. A general linear model was used to detect functional activation (β-value adjusted with family-wise error <0.05, cluster size ≧4). The fractional signal in each voxel was computed by normalizing to the average baseline signal. The relative signal change (ΔS/S) was defined as the difference of fractional signals between resting and activation periods. Temporal SNR (tSNR) was calculated as the signal divided by standard deviation along the time course in each voxel. Contrast-to-noise ratio (CNR) was taken as the product of tSNR and ΔS/S. tSNR and CNR efficiency were defined as tSNR and CNR divided by the square root of acquisition time (in seconds) per slice, respectively, similar to previous studies.

FIG. 2 shows representative images from MPRAGE (anatomical), 3D T2prep-GRE (fMRI scan a) and 2D multi-slice SE EPI (no stimulation, scan d). Geometric distortion is visible in SE EPI images, especially in the frontal and temporal lobes (red arrows). On the other hand, 3D T2prep-GRE images show quite minimal distortion and dropouts across the entire brain. Note that this was achieved with only volume shim to ensure a reasonably homogeneous B0 across the whole brain.

Representative fMRI results from one subject are shown in FIGS. 3A-3C. Robust activation in both visual (mainly row 2) and motor (mainly row 5) cortices was detected with 3D T2prep-GRE (FIG. 3A), which is expected from the simultaneous flashing checkerboard and bilateral finger tapping task. Activations in some other cortical regions such as the anterior temporal (row 2) and posterior parietal (row 6) regions were also observed in this subject, which might be related to visual and sensorimotor responses, or simply the result of large noise in single subject level analysis. The details of these activations are unclear to us and beyond the scope of this methodology study, which certainly warrant further investigation possibly with group level analysis. With the same temporal (TR) and spatial resolution, 2D SE EPI (FIG. 3B) can only cover the visual cortex due to power deposition constraints (SAR). Note that the SE EPI slices here were angled to cover as much cortex as possible and to avoid orbitofrontal cortex, while the SE EPI images shown in FIG. 2 were aligned with the Anterior and Posterior commissure (AC-PC) line. Robust activation was detected in the visual cortex with 2D SE EPI. Similar activation patterns in the visual cortex were observed for these two methods (zoomed in and displayed at the bottom of the panels). The average time courses (FIG. 3C) over common activated voxels in the visual cortex from the two scans were comparable, and their temporal characteristics were in general consistent with those of SE BOLD responses in the literature. The standard deviations in the time courses are greater than those of ΔS/S in Table 2, as they represent intervoxel variations in this subject, while the latter reflect intersubject variations.

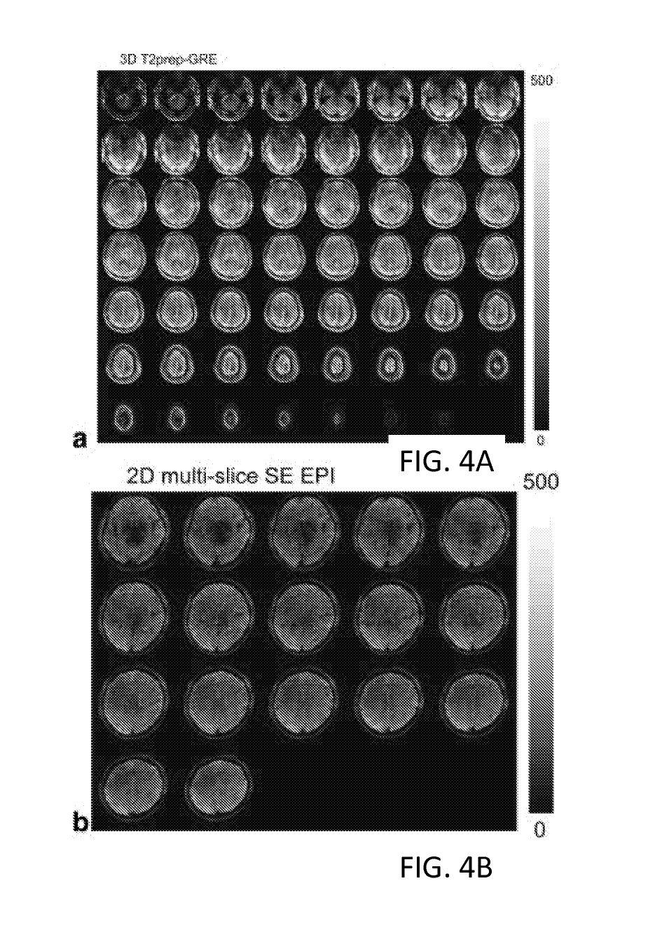

Table 2 summarizes the fMRI results from all subjects (n=5). Slightly more activated voxels (P<0.1) in the visual cortex were detected with 2D SE EPI. When averaging over voxels activated in both scans, relative signal change (ΔS/S), tSNR, CNR and t-score were all slightly higher (P<0.1) in 2D SE EPI, whereas tSNR and CNR efficiency were both significantly greater (P<0.05) in 3D T2prep-GRE. Representative tSNR efficiency maps from both methods are shown in FIGS. 4A and 4B. No spatial smoothing was performed in the initial analysis to minimize its potential influence for the comparison. To show the effects from spatial smoothing, the data was also processed after applying a Gaussian smoothing kernel with a full-width at half maximum (FWHM) of 5 mm. The results remained comparable to those obtained without smoothing, with a slight trend of more activated voxels and smaller ΔS/S in both sequences (not statistically significant, P>0.1). A region-of-interest (ROI) based analysis was also performed, in which signals were averaged over the GM voxels in primary visual and motor cortex, respectively. Similar trends in ΔS/S, tSNR, CNR, t-score, and tSNR and CNR efficiency were observed. Volume shim was applied in all these scans to achieve a reasonably homogeneous B0 across the entire brain and compare the two methods under the same B0 shim condition. As EPI is much more sensitive to B0 inhomogeneity than 3D fast GRE, the SE EPI fMRI scan (b) was repeated with optimal high order shim in the visual cortex. The fMRI results in the visual cortex were comparable to those obtained with volume shim (Table 2). Similar results were also obtained with optimal high order shim in the entire brain (data not shown).

The fMRI scans using 3D fast GRE without T2 preparation (Methods, fMRI scan c) yielded a small number of activated voxels in the whole brain (99±64 for visual and motor cortex combined, n=5). The relative signal changes (ΔS/S) in these scans averaged over all activated voxels in the previous 3D T2prep-GRE scans (Methods, fMRI scan a) were not significantly different from baseline for all five subjects (P>0.1). A typical time course from one subject is shown in FIG. 3C (ΔS/S=0.37±0.57%).

FIG. 5 illustrates fMRI results from one subject using the 3D T2prep-GRE sequence with a voxel size of 1.5×1.5×1.6 mm³, 84 slices and a TR of 1860 ms. Similar to fMRI scan (a) with 2.5 mm isotropic voxel, minimal distortion was seen in the images and robust activation in visual and motor cortices was detected.

It should be noted that the methods described herein can be executed with a program(s) fixed on one or more non-transitory computer readable medium. The non-transitory computer readable medium can be loaded onto a computing device, server, imaging device processor, smartphone, tablet, phablet, or any other suitable device known to or conceivable by one of skill in the art. It should also be noted that herein the steps of the method described can be carried out using a computer, non-transitory computer readable medium, or alternately a computing device, microprocessor, or other computer type device independent of or incorporated with an imaging or signal collection device. The computing device can be integrated with the imaging device for collecting data or can be networked by wire or wirelessly with the imaging device. Indeed, any suitable method of calculation known to or conceivable by one of skill in the art could be used. It should also be noted that while specific equations are detailed herein, variations on these equations can also be derived, and this application includes any such equation known to or conceivable by one of skill in the art. A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A method for magnetic resonance imaging of a subject comprising:
employing a T2-weighted preparation module to induce blood-oxygenation-level-dependent (BOLD) contrast;
providing a single-shot, fast-gradient echo (GRE) readout; and
acquiring an image of the subject.

2. The method of claim 1 further comprising providing the single-shot fast-GRE readout having a short echo time.

3. The method of claim 2 further comprising using the short echo time of approximately <2 ms.

4. The method of claim 1 further comprising using the single shot fast-GRE readout taking the form of at least one of turbo field echo, TFE, or turbo flash.

5. The method of claim 1 further comprising acquiring the image further comprising a whole brain fMRI image with minimal distortion and dropouts.

6. The method of claim 5 further comprising acquiring the image further comprising a spatial resolution of approximately 2.5 mm isotropic.

7. The method of claim 5 further comprising acquiring the image comprising a temporal resolution of 2.3 s at 7 T.

8. The method of claim 1 further comprising generating the BOLD contrast before providing the single-shot, fast GRE readout.

9. The method of claim 1 further comprising using two 180° pulses in the T2-weighted preparation module to compensate for phase variations and to suppress inflow effects.

10. The method of claim 1 further comprising playing a spoiler gradient at an end of the T2-weighted preparation module on a first phase encoding axis that has a lowest gradient duty cycle to dephase any residual transverse magnetization.

11. The method of claim 1 further comprising using a SINC RF pulse for refocusing.

12. The method of claim 1 further comprising using the single-shot fast-gradient echo readout comprising low-high (centric) phase encoding.

13. A system for magnetic resonance imaging comprising:
a magnetic resonance imaging scanner;
a non-transitory computer readable medium programmed to execute steps comprising:
employing a T2-weighted preparation module to induce blood-oxygenation-level-dependent (BOLD) contrast;
providing a single-shot, fast-gradient echo (GRE) readout; and
acquiring an image of the subject.

14. The system of claim 13 wherein the non-transitory computer readable medium is integrated into the magnetic resonance imaging scanner.

15. The system of claim 13 wherein the non-transitory computer readable medium resides on a computing device networked with the magnetic resonance imaging scanner.

16. The system of claim 13 further comprising the single-shot fast-GRE having a short echo time.

17. The system of claim 16 wherein the short echo time is approximately <2 ms.

18. The system of claim 13 wherein the single shot fast-GRE takes the form of at least one of turbo field echo, TFE, or turbo flash.

19. The system of claim 13 wherein the image further comprises a whole brain fMRI image with minimal distortion and dropouts.

20. The system of claim 19 wherein the image further comprises a spatial resolution of approximately 2.5 mm isotropic.

21. The system of claim 19 wherein the image comprises a temporal resolution of 2.3 s at 7 T.

22. The system of claim 13 wherein the BOLD contrast is generated before providing the single-shot, fast GRE readout.

23. The system of claim 13 further comprising using two 180° pulses in the T2-weighted preparation module to compensate for phase variations and to suppress inflow effects.

24. The system of claim 13 further comprising playing a spoiler gradient at an end of the T2-weighted preparation module on a first phase encoding axis that has a lowest gradient duty cycle to dephase any residual transverse magnetization.

25. The system of claim 13 further comprising using a SINC RF pulse for refocusing.

26. The system of claim 13 further comprising using the single-shot fast-gradient echo readout comprising low-high (centric) phase encoding.
